(19) United States
(12) Patent Application Publication
(10) Pub. No.: US 2020/0077929 A1
(43) Pub. Date: Mar. 12, 2020

(54) SYSTEM FOR MONITORING FETAL STATUS DURING CHILD BIRTH

(71) Applicant: ANGEM Devices, Inc, Wyndmoor, PA (US)

(72) Inventors: John S. Missanelli, Wyndmoor, PA (US); Vijay K. Lumba, San Jose, CA (US); Angela K. Lumba, San Jose, CA (US); Megan Missanelli, Wyndmoor, PA (US)

(73) Assignee: ANGEM Devices, Inc, Wyndmoor, PA (US)

(21) Appl. No.: 16/540,615

(22) Filed: Aug. 14, 2019

Related U.S. Application Data
(60) Provisional application No. 62/718,754, filed on Aug. 14, 2018.

(51) Int. Cl. A61B 5/1464; A61B 5/0205; A61B 5/00; A61B 5/1455

(52) U.S. Cl. CPC A61B 5/1464; A61B 5/0205; A61B 2562/0238; A61B 5/14551; A61B 2503/02; A61B 5/4362

(57) ABSTRACT

During childbirth process, trauma to an infant can readily arise, ultimately resulting in fetal hypoxia, academia, and brain damage. Such unfavorable conditions can be prevented by measuring the fetus' blood-oxygen level and heart rate. Without a fetal pulse oximeters, blood oxygen level cannot be monitored non-invasively reliably, which reduces the chance for birth complications to be recognized in time. A noninvasive system to implement such goals and maximize the potential welfare of the fetus may include devices to measure oxygen saturation of hemoglobin (SpO2) that have been available for at least 50 years. Such a device may be an oxy probe that uses a trans-reflective method of SpO2 measurement where oxygen saturation data can be transmitted through wire, fiber optics, and or using a radio frequency link, fetal monitor data can be analyzed, compared to existing data base, and or transmitted via radio waves or internet.

### BACKGROUND

Pulse oximeters have conventionally been used to measure the oxygen saturation of arterial blood continuously. To use the pulse oximeters, a probe is attached to the tip of a subject's finger or earlobe and both red and the probe applies infrared light having different wavelengths to the living body from the probe at given time intervals, and the oximeter calculates the oxygen saturation from the ratio between the RED and IR of light absorbance. In a typical case, the red light has a reference wavelength of 660 nm and the infrared light has a wavelength of 900 nm; two light-emitting diodes of these wavelengths and one photodiode for light reception may be contained in the probe.

Although fetal heart monitors can be used as a surrogate means to attempt to measure fetal blood oxygen saturation levels, this method is indirect, and thus does not give a fully complete understanding of the fetal status. As a result of this lack of full understanding, emergency medical decisions, such as when to start an emergency caesarean section (C-section) must be made with incomplete knowledge. As a practical matter, doctors sometimes err on the side of caution, which may result in unnecessary C-sections, and the attendant high medical expenses and maternal post-childbirth complications.

Previous attempts to provide this missing fetal blood oxygen saturation levels include the OxiFirst system, produced by Mallinckrodt/Nellcor, now part of Tyco Healthcare. This system, which obtained FDA approval in 2000, works by directly placing the tip of a pulse oximeter sensor up the maternal birth canal, through the cervix, into the uterus and onto the cheek or temple of the fetus. This method is described in U.S. Pat. Nos. 5,813,980; 5,109,849, 4,938,218 which are incorporated by reference as if fully set forth herein. Unfortunately, due to the high invasiveness and bother of the procedure, the method met with limited medical acceptance in the field, and the manufacturer eventually decided to stop selling the device.

U.S. Pat. No. 5,135,006, which is incorporated by reference as if fully set forth herein, shows a method and apparatus for monitoring the fetus in a birth canal during labor. This fetal monitor probe monitors heartbeat and does not directly measure the blood oxygenation.

U.S. Pat. No. 10,415,163, which is incorporated by reference as if fully set forth herein, is based on similar pulse oximetry principal for non-invasive monitoring of fetal blood oxygenation by directing light at the abdomen of a pregnant woman, and detecting light scattered and reflected by fetal and maternal tissues back to the surface of the mother's abdomen. It may not be as accurate because of the distance between the probe and the fetus during delivery.

U.S. Pat. No. 7,469,158, which is incorporated by reference as if fully set forth herein, is also based on similar pulse oximetry principal for non-invasive monitoring of fetal blood oxygenation but requires it to be screwed in the scalp. It presents a more invasive technology than is desirable.

U.S. Pat. No. 8,417,307, which is incorporated by reference as if fully set forth herein, relates to a transmissive type blood oximeter for measuring the oxygenation, but it cannot be used in case of fetal measurements.

Thus, there exists a need for a less invasive, accurate, pulse oximeter with a probe that can accurately and safely measure arterial oxygen saturation of a fetus.

### SUMMARY OF THE EMBODIMENTS

During childbirth process, trauma to an infant can readily arise, ultimately resulting in fetal hypoxia, academia, and brain damage. Such unfavorable conditions can be prevented by measuring the fetus' blood-oxygen level and heart rate. Without a fetal pulse oximeters, blood oxygen level cannot be monitored non-invasively reliably, which reduces the chance for birth complications to be recognized in time. A noninvasive system to implement such goals and maximize the potential welfare of the fetus may include devices to measure oxygen saturation of hemoglobin (SpO2) that have been available for at least 50 years. Such a device may be an oxy probe that uses a trans-reflective method of SpO2 measurement where oxygen saturation data can be transmitted through wire, fiber optics, and or using a radio frequency link, fetal monitor data can be analyzed, compared to existing data base, and or transmitted via radio waves or internet.

### BRIEF DESCRIPTION OF THE DRAWINGS

FIG. 1 shows the oxy probe with the connector.

FIG. 2 shows a cross-section A-A of the oxy probe shown in FIG. 1.

FIG. 3 shows a partial exploded view of the oxy probe and its components.

FIG. 4 shows a depiction of the invention in use.

FIG. 5 shows a cut away view of the oxy probe tip illustrating the light path of the reflected light.

FIG. 6 shows a cutaway view of the oxy probe tip.

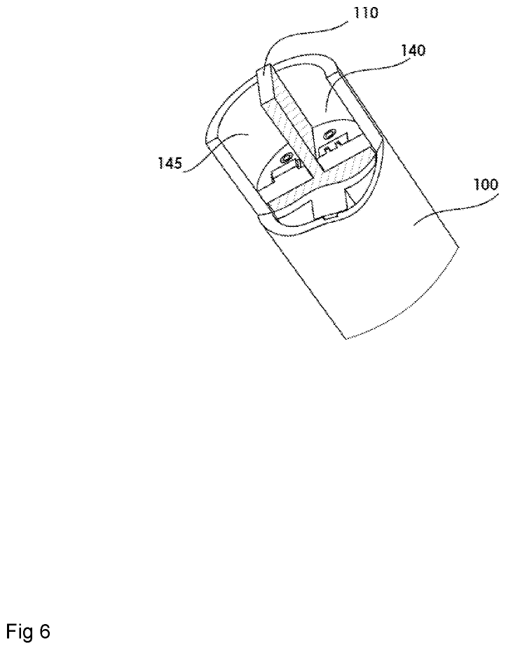

### DETAILED DESCRIPTION OF THE EMBODIMENTS

### Introduction

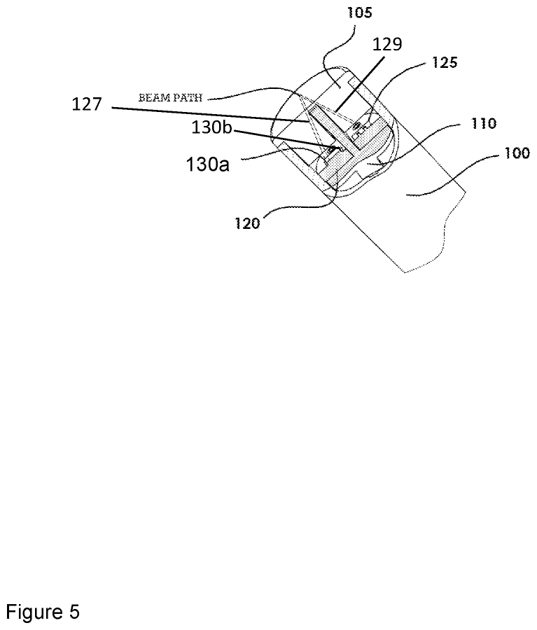

The underling principal of operation is based on the red and infrared light absorption characteristics of oxygenated and deoxygenated hemoglobin. Oxygenated hemoglobin absorbs more infrared light and allows more red light to pass through. Deoxygenated (or reduced oxygen) hemoglobin absorbs more red light and allows more infrared light to pass through. Red light is in the 640-720 nm wavelength light band. Infrared light is in the 840-920 nm wavelength light band. The embodiment includes a cylindrical housing 100 having two different light sources 130a, 130b, collectively 130 (if a single source), that emit emitted light 127 and a detector or sensor 125 with an opaque partition 110 between sources and detector such a way that the detector 125 will see the reflected light 129 as shown in FIG. 5. The probe 100 may include a clear tip or cap 105 that seals the tip from external fluids and at the same time allows the optical signal/light to be transmitted and received with no to minimal attenuation.

In an alternate embodiment, the probe for measuring hemoglobin oxygenation may require two wavelength emitters, Red (640-720 nm) and IR (840-920). Thus, further emitters may be used in this instance using the same probe configuration in which two other wavelengths can be added. An optional feature for detecting proximity to a fetus may be integrated into the probe.

### DETAILED DESCRIPTION

The SpO2 monitoring PROBE (called oxy probe herein) may include, as labeled:

100. Probe body or housing

105. Soft clear probe Tip

110. Probe optical divider

115. Probe wire

120. Integrated circuit IC or PC Board

125. Sensor/detector Chip

127. Emitted light.

129. Reflected or received light.

130a, 130b. Light sources

130. LED Chips

135. Probe Connector

140. Emitter Cavity

145. Sensor Cavity

200. Fetus

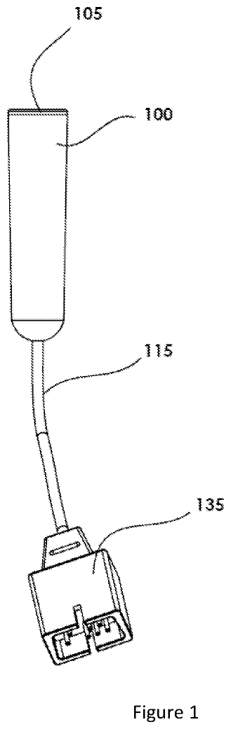

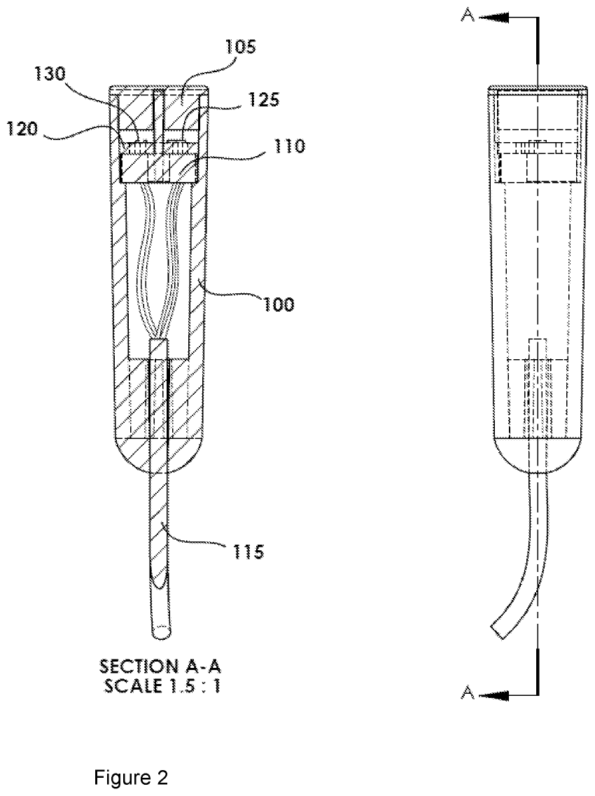

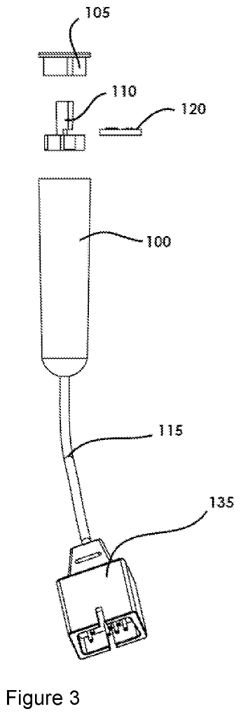

As shown in FIGS. 1-5, an intra-vaginal and intra-uterine oxy probe 100 allows the arterial oxygen saturation of the fetus 200 to be measured during child birth process. The oxy probe 100 has a tip 105 that may be made of soft optically clear silicone type material in its housing and tip 105, which may be safely pressed against the fetus 200 without causing any injury. The tip 105 of the oxy probe 100 is designed to have two optically isolated compartments 140 and 145: One compartment to house the emitters 140 and another to house the sensor 145, wherein the compartments may be separated by an opaque divider 110 that prevents the emitted light 127 from being directly transmitted to the sensor 145. The fetal blood pulse oximetry oxy probe 100 may use at least two wavelengths of emitted light 127 from a first emitter 130a at about 640 to 680 nm and a second wavelength of light at about 870 to 920 nm 130b, wherein the emitters are preferably LED light emitters. The optically clear oxy probe tip 105 allows the emitted light 127 to illuminate fetal tissue and the sensor 125 can detect the reflected light 129. A CPU remote from the tip 105 may perform signal processing to extract the oxygen saturation information related to fetal arterial blood.

The oxy probe may be housed in a housing 100, with dimensions of approximately 0.5 inch in diameter and 2.2-inch-long. A top 0.5 inch of the probe may include the sensor 125, emitters 130 and optical insert 110. These are nominal dimensions and can vary based on requirements, but are chosen to minimize invasiveness to the pregnant woman. The optical divider 110 may be opaque and divide the tip 105 of the probe 100 in two compartments, maintaining the optical isolation between the emitter cavity 140 and sensor 145 cavity (compartments) such that the only way for light to pass between the compartments is through reflection off another surface. The tip of the probe 100 may have a clear, soft, flexible elastomeric material lens 105 that extends into the cavities 140, 145 in such a way as to act as a seal to prevent any fluids from entering the cavities 140, 145 (FIG. 5).

This oxy probe 100 may accommodate additional sensors for monitoring other conditions of a patient, including an arterial hemoglobin oxygen saturation sensor. Most common pulse oximeters used in the hospitals are of transmittance type, whereas the emitters 130s. 130b are placed on one side and the light goes through the tissue to the sensor 125 on the opposite side of the tissue. Alternatively, the emitter 130 and sensor 125 components used in both cases may be similar, with the difference that in the reflective probe, the light reflected by the tissue is compared to light going through the tissue.

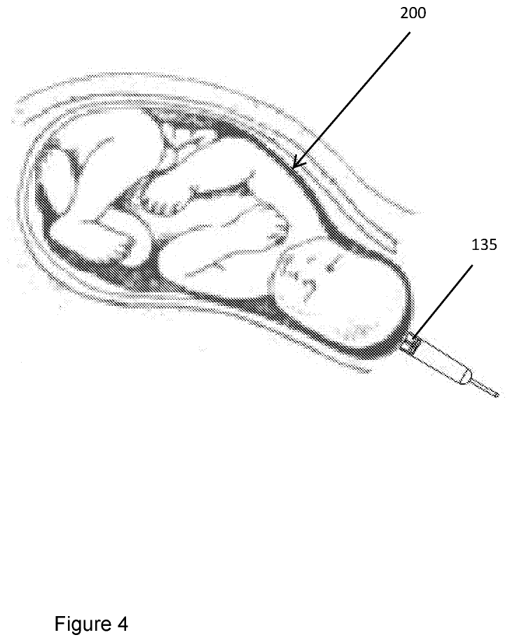

This oxy probe 100 may be used on any location on the body and it does not have to be pressed against the tissue, for example, close (<1.0 mm) contact with the surface renders accurate data. For monitoring a fetus 200, the oxy probe 100 may be applied through a dilated cervix. The oxy probe 100 may monitor the condition of a fetus 200 during the peripartum process, measuring fetal heart rate, arterial hemoglobin oxygen saturation, electrical activity of the heart, or a combination thereof, by touching the scalp of the fetus 200 (as shown in FIG. 4) or any fetal presenting part.

The optical divider 110 may be made of an opaque material that prevents transmission of light reaching the sensor 125 compartment directly from the light source 130. The housing 100 of the probe may be made of a light reflecting color such as white.

The probe tip 105, as mentioned above, may be made of optically clear soft silicon or similar materials. This clear tip 105 enables the emitted light 127 from the light source 130 to reach fetal tissue with minimal loss and allows the light to reflect back as reflected light 129 to the sensor 125 efficiently. The tip 105 being soft, also seals the probe tip such that no fluids can reach the emitter 130 and sensor 125. The sealed probe 100 can function properly to obtain accurate readings even on wet surfaces as well as when completely immersed in a fluid.

The light source 130 can include two or more light emitting diodes (LED) configured to emit light at a selected wavelength. When including more emitters, additional emitters may include a third light emitter that emits an emitted light of 550 nm to 620 nm to enable detection of other tissue bio parameters.

The detector 135 may include one or more silicon photodiodes that produce current linearly proportional to the intensity of light striking it. The detector 135 can detect the absorption and/or scattering of the light from the tissue as well as the frequency of the light emitted from the light source 130.

Unlike conventional pulse oximeters, the devices described herein need not be in direct contact with the patient's skin in order to obtain an accurate, consistent reading also due to their being highly directional and having very high gain. As mentioned above, the oxy probe can be positioned 1 mm or less away from the skin surface and still obtain accurate oxygen saturation and heart beat readings. The probe need not be mechanically coupled to the body to obtain an accurate reading. Because the device need not be in direct contact with the skin and there is no need for mechanical coupling to a patient, the problems that can result including pressure point injuries, pressure necrosis, exsanguinations, discomfort, compression marks, erroneous measurements, infections and other issues caused by direct contact with a device can be avoided.

While the invention has been described with reference to the embodiments above, a person of ordinary skill in the art would understand that various changes or modifications may be made thereto without departing from the scope of the claims.

1. An oximeter probe comprising:
a housing defining a first cavity and a second cavity;
the first cavity comprising at least two light emitters, wherein each emitter emits an emitted light of a different wavelength than the other of the emitters;
the second cavity including a detector for detecting wavelength of reflected light, wherein the reflected light is a portion of emitted light after the emitted light has been reflected off of fetus; and
a divider located between the cavities that prevents the emitted light from being detected by the detector;
a transparent cap that seals a tip of the housing from external fluids and allows the emitted light to be transmitted and received;
wherein a CPU determines oxygen saturation in the fetus based on a difference between the emitted light wavelength and the reflected wavelength.

2. The oximeter probe of claim 1, wherein the two light emitters are LED light emitters.

3. The oximeter probe of claim 2, wherein the light emitters include a first light emitter that emits light with a wavelength of 640 nm to 680 nm and a second light emitter emits light with a wavelength of 870 nm to 920 nm.

4. The oximeter probe of claim 3, further comprising a third light emitter that emits an emitted light of 550 nm to 620 nm to enable detection of other tissue bio parameters.

5. The oximeter probe of claim 1, wherein the transparent cap that allows the emitted light to reach the fetus and receive the reflected light to the detector.

6. The oximeter probe of claim 5, wherein the transparent cap is made from a flexible material.

7. The oximeter probe of claim 6, wherein flexible material comprises an elastomeric material.

8. The oximeter probe of claim 6, wherein the transparent cap extends into the first and second cavities.

9. The oximeter probe of claim 8, wherein the transparent cap acts as a watertight seal to prevent fluid ingress into the first and second cavities.

10. The oximeter probe of claim 6, wherein the oximeter probe can measure the oxygen saturation in the fetus from a distance.

11. The oximeter probe of claim 10, wherein the distance is less than 1 mm.

12. The oximeter probe of claim 1, wherein the housing is 0.5 inches in diameter.

13. The oximeter probe of claim 1, wherein the housing is 2.2 inches long.

14. The oximeter probe of claim 1, wherein the detector comprises silicon photodiodes that produce current linearly proportional to an intensity of the reflected light received at the detector.

15. The oximeter probe of claim 1, wherein the detector detects absorption and/or scattering of the reflected light from the fetus.

16. A method for detecting oxygen saturation in a fetus comprising:
providing an oximeter probe comprising:
a housing defining a first cavity and a second cavity;
the first cavity comprising at least two light emitters, wherein each emitter emits an emitted light of a different wavelength than the other of the emitters;
the second cavity including a detector for detecting wavelength of reflected light, wherein the reflected light is a portion of emitted light after the emitted light has been reflected off of human tissue;
a transparent cap that seals a tip of the housing from external fluids and allows the emitted light to be transmitted and received; and
a divider located between the cavities that prevents the emitted light from being detected by the detector;
placing the oximeter in proximity to the fetus;
determining, using a CPU, oxygen saturation in the fetus based on a difference between the emitted light wavelength and the reflected wavelength.

17. The method of claim 16, wherein the transparent cap that allows the emitted light to reach the fetus and receive the reflected light back to the detector.

18. The method of claim 17, wherein the transparent cap extends into the first and second cavities, and wherein the transparent cap acts as a watertight seal to prevent fluid ingress into the first and second cavities.

19. The method of claim 16, wherein the oximeter probe can measure the oxygen saturation in the fetus from a distance.

20. The method of claim 19, wherein the distance is less than 1 mm.
